(19) United States
(12) Patent Application Publication
(10) Pub. No.: US 2006/0252823 A1
(43) Pub. Date: Nov. 9, 2006

(54) Isocoumarin-based inhibitors of urokinase-type plasminogen activator

(76) Inventors: Lorraine M. Deck, Albuquerque, NM (US); David L. Vander Jagt, Albuquerque, NM (US); Justin J. Heynekamp, Albuquerque, NM (US)

Correspondence Address: Henry D. Coleman, 714 Colorado Avenue, Bridgeport, CT, 06605-1601, US

(21) Appl. No.: 11/360,193

(22) Filed: Feb. 23, 2006

Related U.S. Application Data
(60) Provisional application No. 60/677,775, filed on May 4, 2005.
(60) Provisional application No. 60/677,773, filed on May 4, 2005.

(51) Int. Cl. A61K 31/366; C07D 311/02

(52) U.S. Cl. 514/457; 549/283; 514/309; 549/23

(57) ABSTRACT

The present invention relates to chemical compounds, pharmaceutical compositions and methods for treating tumors and cancer and diseases which involve angiogenesis including retinopathy, age-related macular degeneration, angiogenic skin disorders and inflammation, including chronic inflammatory diseases, such as psoriasis, acne, rosacea, warts, eczema, hemangiomas, lymphangiogenesis, arthritis, lupus and scleroderma, among others. Compounds according to the present invention have the chemical structure: 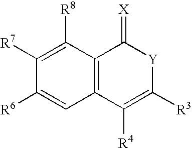 Where X is O or S, preferably O; Y is O, S, or N, preferably O;
R³ is an optionally substituted C₁-C₇ alkyl group, an optionally substituted (CH₂)ₙR^{b} group or an OR group;
R^{b} is a guanidino group or a thioguanidino group;
R is an optionally substituted C₁-C₇ alkyl group or an optionally substituted (CH₂)ₙR′ group; n is 0, 1, 2, 3, 4, 5, 6, or 7 (preferably 2, 3 or 4);
R′ is F, Cl, Br or I (preferably Br), NO₂, an R″ group, an OR″ group or an SR″ group, where R″ is an optionally substituted C₁-C₆ alkyl group, a guanidino group or a thioguanidino group;
R⁴ is H, F, Cl, Br, I, NO₂, OH, R¹ or OR¹, where R¹ is an optionally substituted C₁-C₇ alkyl group or an optionally substituted C₂-C₁₁ acyl group;
R⁶ is H, an optionally substituted C₁-C₆ alkyl group, or together with R⁷ forms an optionally substituted 5-7 membered saturated or unsaturated carbocyclic group, an optionally substituted 5-7 membered saturated or unsaturated heterocyclic group, or an optionally substituted aromatic or heteroaromatic group;
R⁷ is H, F, Cl, Br, I, NO₂, NR^{a′}R^{b′} or NHR^{b}, where R^{a′} and R^{b′} are independently H or a C₁-C₃ alkyl group and R^{b} is a C₂-C₁₁ acyl group which is optionally substituted, or together with R⁶ or R⁸ forms an optionally substituted 5-7 membered saturated or unsaturated carbocyclic group, an optionally substituted 5-7 membered saturated or unsaturated heterocyclic group, or an optionally substituted aromatic or heteroaromatic group;
R⁸ is H, an optionally substituted C₁-C₆ alkyl group, or together with R⁷ forms an optionally substituted 5-7 membered saturated or unsaturated carbocyclic group, an optionally substituted 5-7 membered saturated or unsaturated heterocyclic group, or an optionally substituted aromatic or heteroaromatic group; and pharmaceutically acceptable salts, thereof.

### RELATED APPLICATIONS

This application claims the benefit of priority of provisional application Ser. No. US60/677,773, filed May 4, 2005, the entire contents of which is incorporated by reference herein.

This work was supported by a grant from the National Institutes of Health, number HL68598. Consequently, the government retains certain rights in the invention.

### FIELD OF THE INVENTION

The present invention relates to chemical compounds, pharmaceutical compositions and methods for treating tumors and cancer and diseases which involve angiogenesis including retinopathy, age-related macular degeneration, angiogenic skin disorders and inflammation, including chronic inflammatory diseases, such as psoriasis, acne, rosacea, warts, eczema, hemangiomas, lymphangiogenesis, arthritis, lupus and scleroderma, among others.

### BACKGROUND OF THE INVENTION

Multiple proteases, including matrix metalloproteases (MMP-2, MMP-9 and MMP-14), cysteine proteases (cathepsin B and cathepsin L), aspartyl protease (cathepsin D) and serine proteases (plasmin, matriptase and urokinase) participate in cancer cell growth, metastasis and angiogenesis.¹⁻⁴ High expression of proteases often correlates with a poor prognosis.^{5,6} Urokinase plasminogen activator (uPA) plays an especially important role in extracellular proteolysis that contributes to cancer cell metastasis. Many cancer cells secrete pro-uPA and its receptor uPAR; binding of pro-uPA to uPAR leads to its activation, with subsequent generation of plasmin by the uPA-catalyzed hydrolysis of extracellular plasminogen.^{7,8} The increased production of plasmin leads to degradation of extracellular matrix both by plasmin itself and by other proteases that are activated by plasmin. The surface location of bound uPA provides directionality to the degradation of matrix, thereby assisting the directional migration of cancer cells. uPA in complex with uPAR also affects other biological processes including signaling pathways that influence cell proliferation.⁹ uPA has become a major target for development of non-peptidic small molecule inhibitors as potential anti-cancer drugs.^{10,11}

Most of the efforts to develop potent and selective inhibitors of uPA have focused on arginino mimetics based upon the trypsin-like specificity of uPA. The development of selective inhibitors of uPA is a challenge due to the large number of serine proteases with trypsin-like specificity, including factor VII, factor X and tissue-type plasminogen activator. Extensive structure-based drug development has provided potent and selective inhibitors of uPA; these generally are arginino mimetics with amidine or guanidine functional groups built onto aromatic or heterocyclic scaffolds (FIG. 1).¹²⁻¹⁶ A major limitation to the use of these inhibitors is their poor bioavailability owing to the presence of the positively charged amidine or guanidine groups. This has limited clinical studies of these uPA inhibitors.

In the present invention, we have focused on the synthesis and testing of uncharged compounds as leads for the development of uPA inhibitors with improved bioavailability. 4-Chloroisocoumarin was selected as the scaffold, in which substituted 3-alkoxy groups were introduced that contained neutral terminal functional groups or charged terminal functional groups.¹⁷ Additional substituents were introduced into the seven position. 4-Chloroisocoumarin scaffolds have been used in studies of serine protease inhibitors,¹⁷ but with limited application to uPA.¹⁸ The choice of the 4-chloroisocoumarin scaffold was based upon the potential of these compounds to function as mechanism-based inactivators.¹⁷ In this study we demonstrate that introduction of bromine in place of a terminal charged functional group in the 3-alkoxy substituent provides uncharged uPA inhibitors with low micromolar dissociation constants. Further introduction of substituents at the seven position of these uncharged uPA inhibitors provides compounds with low nanomolar dissociation constants. These inhibitors may serve as lead compounds for the development of new uPA inhibitors. Molecular modeling with human uPA suggests that the bromine occupies the same site as the arginino mimetic functional groups.

### SUMMARY OF THE INVENTION

The invention relates to compounds according to the formula: 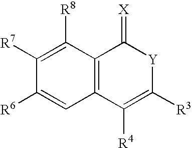
Where X is O or S, preferably O;
Y is O, S, or N, preferably O;
R³ is an optionally substituted CI-C₇ alkyl group, an optionally substituted (CH₂)ₙR^{b} group or an OR group;
R^{b} is a guanidino group or a thioguanidino group;
R is an optionally substituted C₁-C₇ alkyl group or an optionally substituted (CH₂)ₙR^{b} group;
n is 0, 1, 2, 3, 4, 5, 6, or 7 (preferably 2, 3 or 4);
R′ is F, Cl, Br or I (preferably Br), NO₂, an R″ group, an OR″ group or an SR″ group, where R″ is an optionally substituted C₁-C₆ alkyl group, a guanidino group or a thioguanidino group;
R⁴ is H, F, Cl, Br, I, NO₂, OH, R¹ or OR¹, where R¹ is an optionally substituted C₁-C₇ alkyl group or an optionally substituted C₂-C₁₁ acyl group;
R⁶ is H, an optionally substituted C₁-C₆ alkyl group, or together with R⁷ forms an optionally substituted 5-7 membered saturated or unsaturated carbocyclic group, an optionally substituted 5-7 membered saturated or unsaturated heterocyclic group, or an optionally substituted aromatic or heteroaromatic group;
R⁷ is H, F, Cl, Br, I, NO₂, NR^{a′}R^{b′} or NHR^{b}, where R^{a′} and R^{b′} are independently H or a C₁-C₃ alkyl group and R^{b} is a C₂-C₁₁ acyl group which is optionally substituted, or together with R⁶ or R⁸ forms an optionally substituted 5-7 membered saturated or unsaturated carbocyclic group, an optionally substituted 5-7 membered saturated or unsaturated heterocyclic group, or an optionally substituted aromatic or heteroaromatic group;
R⁸ is H, an optionally substituted C₁-C₆ alkyl group, or together with R⁷ forms an optionally substituted 5-7 membered saturated or unsaturated carbocyclic group, an optionally substituted 5-7 membered saturated or unsaturated heterocyclic group, or an optionally substituted aromatic or heteroaromatic group; and pharmaceutically acceptable salts, thereof.

In preferred aspects of the present invention, compounds according to the present invention are uncharged.

The present invention also relates to pharmaceutical compositions, including quick release or sustained/controlled release comprising an effective amount of at least one compound as otherwise described herein in combination with a pharmaceutically acceptable carrier, additive or excipient.

The present invention also relates to methods for treating tumors, including cancer, as well as a number of disease states or conditions which are modulated through an angiogenesis mechanism. These disease states or conditions include retinopathy, age-related macular degeneration, angiogenic skin disorders and inflammation, including chronic inflammatory diseases, such as psoriasis, acne, rosacea, warts, eczema, hemangiomas, lymphangiogenesis, arthritis, lupus and scleroderma, among others. The methods of the present invention comprise administering a therapeutically effective amount of one or more of the disclosed compounds to a patient in need of such treatment.

### BRIEF DESCRIPTION OF THE FIGURES

FIG. 1 shows some basic chemical scaffolds that have been utilized to develop arginino mimetic uPA inhibitors in the amidine (I-VI) and guanidine (VII-IX) series.¹²-16

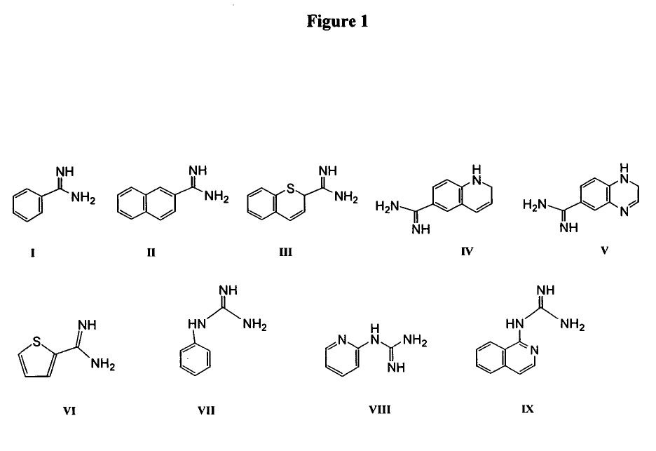

FIG. 2 shows a molecular model of human uPA complexed with inhibitor 8b. The bromine is within 3.32 Å of aspartic acid 189.

FIG. 3 shows a molecular model of human uPA complexed with inhibitor 9b. The isothiourea is within 2.02 Å of aspartic acid 189.

FIG. 4 shows the dissociation constants for inhibition of human uPA in Table 1.

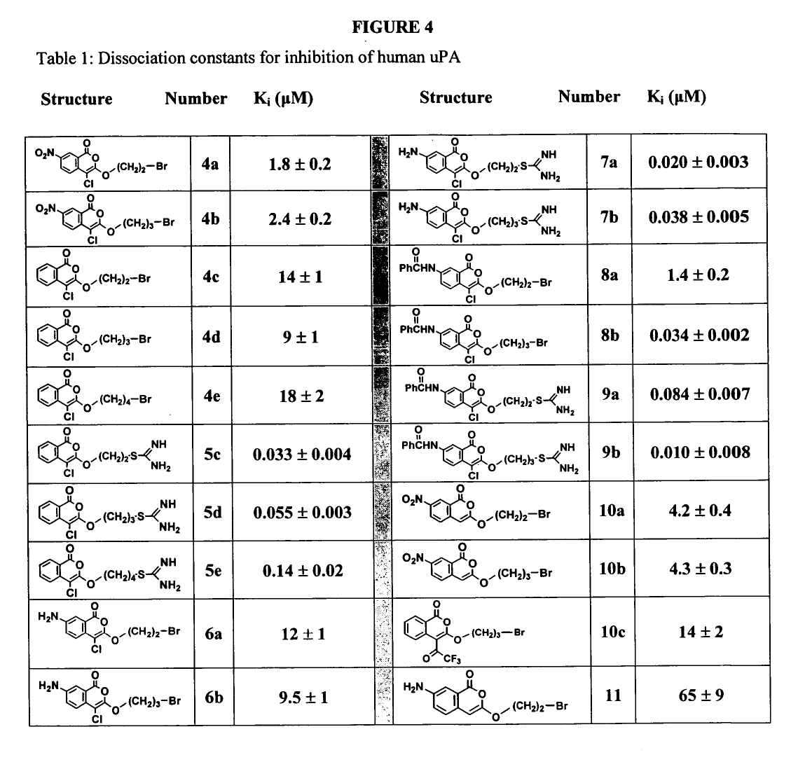

FIG. 5 shows scheme I and scheme II, which relate to synthetic chemical steps for making identified compounds according to the present invention.

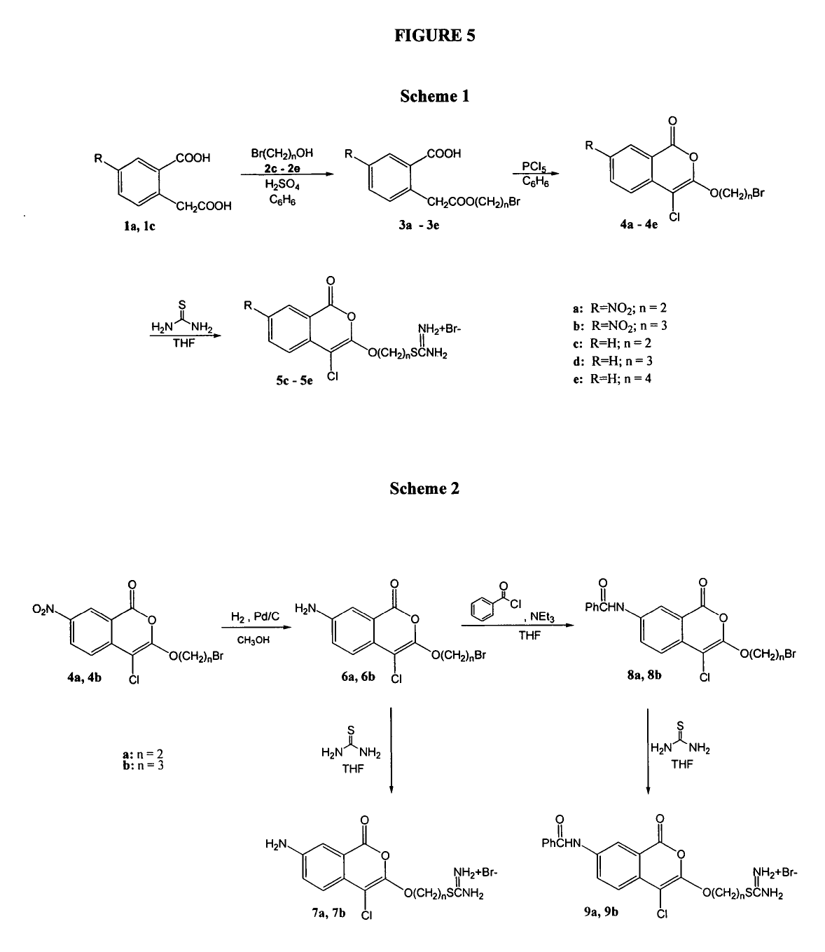

FIG. 6 shows scheme II, which relates to synthetic chemical steps for making identified compounds according to the present invention.

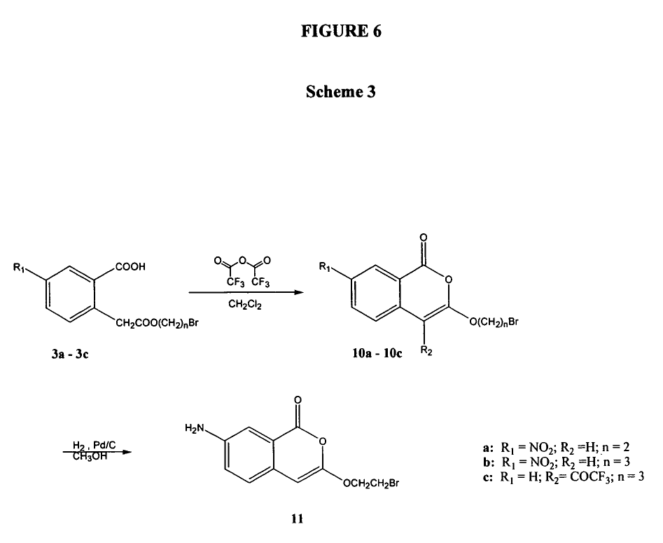

### DETAILED DESCRIPTION OF THE INVENTION

The term patient is used throughout the specification to describe an animal, preferably a human, to whom treatment, including prophylactic treatment, with the compounds according to the present invention is provided. For treatment of those infections, conditions or disease states which are specific for a specific animal such as a human patient, the term patient refers to that specific animal. In most instances, the term patient refers to a human patient.

The term “effective amount” is used throughout the specification to describe concentrations or amounts of compounds according to the present invention which may be used to produce a favorable change in the disease or condition treated, whether that change is a remission, a decrease in growth or size of cancer, tumor or other growth, a favorable physiological result including the clearing up of skin or tissue, or the like, depending upon the disease or condition treated.

The term “neoplasia” is used throughout the specification to describe the pathological process that results in the formation and growth of a neoplasm, i.e., an abnormal tissue that grows by cellular proliferation more rapidly than normal tissue and continues to grow after the stimuli that initiated the new growth cease. Neoplasia could be a distinct mass of tissue that may be benign (benign tumor) or malignant (carcinoma). As used herein, the term neoplasia is used to describe all cancerous disease states and embraces or encompasses the pathological process associated with malignant hematogenous, ascitic, and solid tumors.

Cancer which may be treated using compositions according to the present invention include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, melanoma, acute lymphocytic leukemia, acute myelogenous leukemia, Ewing's sarcoma, small cell lung cancer, choriocarcinoma, rhabdomyosarcoma, Wilms' Tumor, neuroblastoma, hairy cell leukemia, mouth/pharynx, oesophagus, larynx, kidney, lymphoma, among others. The treatment of tumors comprising administering to a patient an anti-tumor effective amount of one or more these agents is a preferred embodiment of the present invention.

The term angiogenesis is used throughout the specification to describe the biological processes which result in the development of blood vessels or increase in the vascularity of tissue in an organism. With respect to the present invention, the term angiogenesis is defined as the process through which tumors or other rapidly proliferating tissue derive a blood supply through the generation of microvessels.

The terms angiogenic disease, angiogenic disorder and angiogenic skin disorder is used throughout the specification to describe a disorder, generally a skin disorder or related disorder which occurs as a consequence of or which results in increased vascularization in tissue. Oftentimes, the etiology of the angiogenic disease is unknown. However, whether angiogenesis is an actual cause of a disease state or is simply a condition of the disease state is unimportant, but the inhibition of angiogenesis in treating or reversing the disease state or condition is an important aspect of the present invention. Examples of angiogenic skin disorders which may be treated utilizing compounds according to the present invention include, for example, psoriasis, venous ulcers, acne, rosacea, warts, eczema, hemangiomas and lymphangiogenesis, among numerous others, including Sturge-Weber syndrome, neurofibromatosis, tuberous sclerosis, chronic inflammatory disease and arthritis. Any skin disorder which has as a primary or secondary characterization, increased vascularization, is considered an angiogenic skin disorder for purposes of the present invention and is amenable to treatment with compounds according to the present invention.

The term “substituted” shall mean substituted at a carbon (or nitrogen) position with, in context, one or more hydroxyl, carboxyl, halogen (F, Cl, Br, I), an alkyl group (preferably, C₁-C₆), alkoxy group (preferably, C₁-C₆ alkyl or aryl), ester (preferably, C₁-C₆ alkyl or aryl) including alkylene ester (such that attachment is on the alkylene group, rather than at the ester function which is preferably substituted with a C₁-C₆ alkyl or aryl group) and cyano. Preferably, the term “substituted” shall mean within its context of use alkyl, alkoxy, halogen, hydroxyl, carboxylic acid and cyano. The term unsubstituted shall mean substituted with one or more H atoms.

“Aromatic” refers to a substituted or unsubstituted monovalent aromatic (aryl) radical having a single ring (e.g., benzene) or multiple condensed rings (e.g., naphthyl, anthracenyl, phenanthryl). Heterocyclic aromatic ring systems “heteroaromatic” groups having one or more nitrogen, oxygen, or sulfur atoms in the ring, such as imidazole, furyl, pyrrole, pyridyl, indole and fused ring systems, among others, which may be substituted or unsubstituted are also contemplated for use in the present invention.

The term “cyclic” shall refer to a carbocyclic or heterocyclic group as indicated in the text, having one to three rings, preferably one ring which is preferably a 5-7 membered ring, more preferably a 5 or 6 membered ring. A heterocyclic ring shall contain at least one atom other than carbon selected from nitrogen, sulfur and oxygen. These cyclic groups may be saturated or unsaturated.

The following examples provide embodiments according to the present invention but are not to be limited in any way.

Chemistry

Compounds 4a-4e, which are 3-bromoalkoxy-4-chloroisocoumarins, were synthesized as shown in Scheme 1. Two of the compounds, 4a and 4b, have a nitro group in position seven. These compounds have varying lengths of bromoalkoxy groups tethered in position three of the isocoumarin scaffold. 5-Nitrohomophthalic acid (1a) was prepared by regioselective nitration of homophthalic acid (1c) using fuming nitric acid by the method described by Ungnade, Nightingale, and French.¹⁹ 5-Nitrohomophthalic acid (1a) and homophthalic acid (1c) were monoesterified using bromoalcohols, compounds 2c-2e, in the presence of sulfuric acid to give moderate yields of bromoesters, 3a-3e. Monoesterification at the saturated acid has been attributed to the mesomeric effect of the carboxyl with the double bond in the aryl ring by Devi and Rajaram.²⁰ Cyclization of the esters, compounds 3a-3e, with phosphorus pentachloride in toluene gave 3-bromoalkoxy-4-chloroisocoumarins, 4a-4e, in moderate yields using a variation of the method described by Powers and coworkers.¹⁷ Compounds 4a-4e were synthesized to test the importance of an uncharged group in the three position of 4-chloroisocoumarins and the length of the tether of the alkoxy group.

Compounds 5c-5e are 4-chloro-3-isothioureidoalkoxyisocoumarin salts and were synthesized as shown in Scheme 1. Nucleophilic substitution of the bromine in compounds 4c-4e was achieved by refluxing these compounds with thiourea in tetrahydrofuran to give the hydrobromide salts, compounds 5c-5e, in moderate yields. The 7-nitrosubstituted isocoumarins, compounds 4a and 4b, did not give any identifiable products when reacted with thiourea in tetrahydrofuran. Compounds 5c-5e were synthesized to test the importance of a charged alkoxy group in the three position of 4-chloroisocoumarins.

The synthesis of 7-amino-3-bromoalkoxy-4-chloroisocoumarins, compounds 6a and 6b, is shown in Scheme 2. These 7-aminoisocoumarins were prepared by reduction of 3-bromoalkoxy-4-chloro-7-nitroisocoumarins, compounds 4a and 4b, using hydrogen in the presence of a catalytic amount of 10% palladium on charcoal under pressure in methanol. Compounds 6a and 6b were synthesized to test the importance of an amino group in the seven position and an uncharged alkoxy group in the three position of 4-chloroisocoumarins. Nucleophilic substitution of the bromine in compounds 6a and 6b by reaction with thiourea afforded hydrobromide salts, 7a and 7b. Compounds 7a and 7b were synthesized to test the importance of a charged alkoxy group in the three position of 7-amino-4-chloroisocoumarins.

7-Benzamido-3-bromoalkoxy-4-chloroisocoumarins, compounds 8a and 8b, were synthesized by reaction of 6a and 6b with benzoyl chloride in the presence of triethylamine (Scheme 2). Compounds 8a and 8b were synthesized to test the importance of an uncharged alkoxy group in the three position and a hydrophobic benzamide group in the seven position of 4-chloroisocoumarins. The two amides, 8a and 8b were reacted with thiourea to give the hydrobromide salts, 9a and 9b, in moderate yields. Compounds 9a and 9b were synthesized to test the importance of a charged alkoxy group in the three position and a hydrophobic benzamide group in the seven position of 4-chloroisocoumarins.

Scheme 3 describes the synthesis of two 3-bromoalkoxy-7-nitrosoisocoumarins, compounds 10a and 10b, which do not have a chlorine atom at position four. The synthesis of compound 10c, which is a 3-alkoxy-4-trifluoroacetylisocoumarin, is also described in Scheme 3. Cyclization of compounds 3a and 3b, which have a nitro group in position seven, using trifluoroacetic anhydride gave compounds 10a and 10b, which contain a hydrogen at position four. On the other hand, cyclization of 3c which does not contain a nitro group in position seven gave compound 10c, which contains a trifluoroacetyl group in position four. This may be attributed to the fact that the intramolecular cyclization of the initially formed enol is faster when there is resonance stabilization by the electron withdrawing nitro group. When the nitro group is absent the initially formed enol reacts with trifluoroacetic anhydride giving compound 10c. Compounds 10a and 10b were synthesized to test the importance of a chlorine atom in position four and the presence of an electron withdrawing group in position seven of the isocoumarins. Compound 10c gives information on the importance of a trifluoroacetyl group in position four of the isocoumarins.

Compound 11, a 7-amino-3-bromoethoxyisocoumarin, was prepared by reduction of compound 10a using hydrogen in the presence of a catalytic amount of 10% palladium on charcoal under pressure in methanol (Scheme 3). Compound 11 was synthesized to test the importance of a chlorine atom in position four and the presence of an electron donating group in position seven of the isocoumarins.

Results and Discussion

On the basis of docking studies using the crystal structure of human uPA¹⁵ we examined whether the 4-chloroisocoumarin scaffold containing 3-alkoxy substituents is predicted to be a good template for the design of uPA inhibitors. Our interest focused on compounds having an uncharged bromine group in place of the charged arginino mimetic group at the terminal position of the 3-alkoxy group. Specifically, we compared the experimentally determined dissociation constants with the docking orientations predicted for compounds with the isocoumarin scaffold containing a charged isothiourea group or an uncharged bromine atom in the terminal 3-alkoxy position.

The first series of compounds compared the 4-chloroisocoumarin scaffold with 3-alkoxy substituents where the terminal functional group was an isothiourea group or bromine atom. The distance between the terminal group and the alkoxy oxygen was varied by the insertion of methylene units (Table 1). The designed inhibitors were shown to dock in the area of the active site containing the catalytic triad (serine 195, histidine 57, aspartic acid 102) and aspartic acid 189, which is the residue in trypsin-like enzymes that forms a salt bridge with the arginino mimetic groups. All of the compounds were predicted to reside in the active site with the thiourea group and the bromine atom in the S1-subsite which contains aspartic acid 189. Compounds 4c-4e, which are 3-bromoalkoxy-4-chloroisocoumarins, are oriented in the active site with the uncharged bromine atom within 2.81-3.12 Å of aspartic acid 189. The best inhibitor in this series, compound 4d, exhibited Kᵢ=9 μM. Modeling of inhibitor 4d with uPA also showed that three methylene units between the bromine and oxygen of the 3-alkoxy substituent provided the closest interaction between the isocoumarin carbonyl and the active site residue serine 195. Nevertheless, compound 4d provided simple competitive inhibition with no evidence of rapid inactivation of uPA by 4d. For comparison, compounds 5c-5e were included in this series. These compounds contain the charged isothiourea group in place of the bromine atom and two, three or four methylene units between the isothiourea group and the oxygen atom. Modeling suggests that compounds 5c-5e are all oriented in the active site with the charged isothiourea group forming a salt bridge with aspartic acid 189. The predicted distance between the isothiourea group and aspartic acid 189 for compounds 5c-5e ranges from 2.49- 2.58 A. The best inhibitor, compound 5c, exhibited a Kᵢ=0.027 μM. Modeling suggested that the 3-alkoxy substituent with two methylenes between the isothiourea group and oxygen atom provided the closest interactions between the isocoumarin carbonyl and the active site serine. Clearly, the charged isothiourea group provides 3.5-4 kcal/mol more binding energy than the bromine atom in its interactions with aspartic 189 and surrounding residues. Thus, there is a large penalty in moving to the uncharged bromoalkoxy inhibitors. This may be the penalty that must be accepted if uncharged uPA inhibitors with improved bioavailability are to be developed in place of the charged uPA inhibitors that have been described.¹²⁻¹⁶

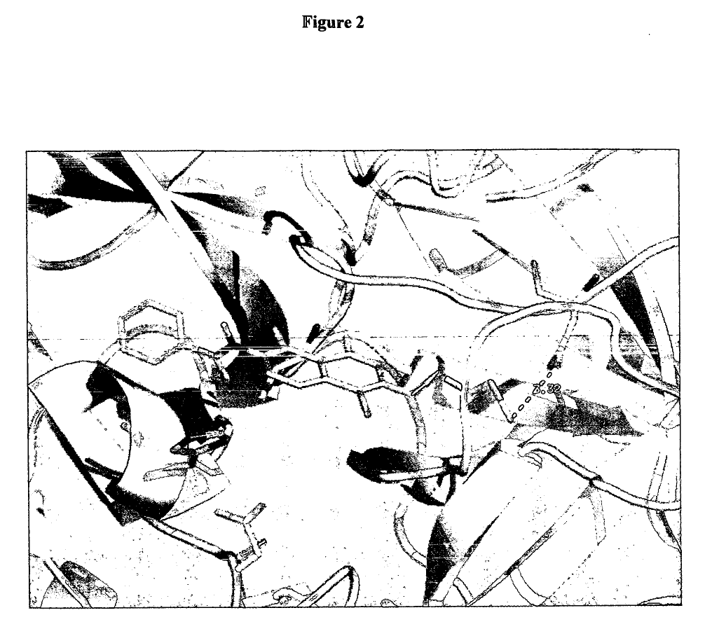

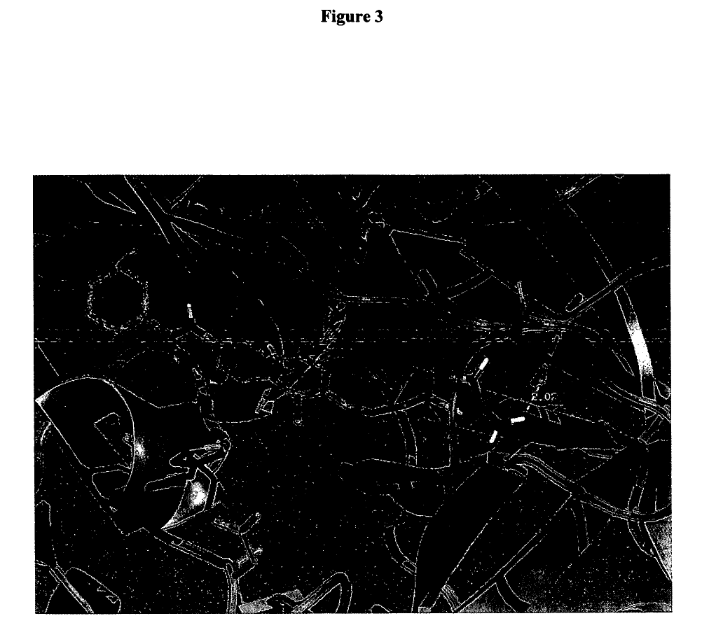

The second series of uPA inhibitors examined the effects of substituents in the seven position. Compounds 6a, 6b, 7a and 7b, which are 7-amino-4-chloroisocoumarins are attractive candidates because of the increased hydrogen bonding possibilities. Based upon modeling, compounds 6a and 6b, which have an uncharged bromine atom on the 3-alkoxy group, are within 2.80-3.15 Å of aspartic acid 189. Compounds 7a and 7b, which have a charged isothiourea group within 2.75-2.85 Å from aspartic acid 189. Compounds 8a, 8b, 9a and 9b are 7-substituted benzamides that were synthesized to investigate potential hydrophobic interactions from groups at the seven position. The uncharged bromine atoms of compounds 8a and 8b are within 2.93-3.32 Å of aspartic acid 189. The charged isothiourea group in compounds 9a and 9b are within 2.75 Å of aspartic acid 189. In the isothiourea series (7a, 7b, 9a and 9b), the 7-substituted compounds did not exhibit any significant improvement in binding to uPA (Table 1). By comparison, the compounds in the bromo series with hydrophobic groups at the seven position (8a and 8b) exhibited markedly improved binding; for 8b, Kᵢ=0.034 μM, which represents a 300-fold improvement over 4d. Inhibitor 8b was the best inhibitor in the bromo series and can be viewed as a promising lead compound for development of uncharged inhibitors of uPA. The improvement that the benzamide group in 8a and 8b of the bromo series provided compared to the lack of improvement that the benzamide group provided in the isothiourea series may relate to increased flexibility. The bromo group has weak interactions with aspartic acid 189; this may allow the inhibitors in the bromo series to slide within the active site and S1 sub-site to orientations that allow the 7-benzamide group to find additional energetically favorable interactions. By comparison, the strong salt bridge in the uPA complexes with inhibitors in the isothiourea series may lock the inhibitors into the S1 sub-site. The modeled uPA complexes with inhibitors 8b and 9b are shown in FIGS. 2 and 3 respectively.

The presence of a 7-nitro group also was beneficial. Compounds 4a and 4b, which are 3-bromoalkoxy-7-nitroisocoumarins, showed improved binding to uPA compared to the unsubstituted compounds. Compound 4b which has three methylene units between the bromine atom and the oxygen of the 3-alkoxy group has a Kᵢ=1.2 μM, which is an 8-fold improvement over the unsubstituted compound 4d.

Compounds 10a, 10b and 11 were synthesized to determine whether the chlorine atom in position four contributed to binding. 3-Bromoalkoxy-7-substituted isocoumarins without a chlorine atom in the four position show dissociation constants that are 3-5 fold higher compared to their counterparts that have a chlorine atom in the four position suggesting a modest role for the chlorine atom (Table 1). Compound 10c which has trifluoroacetyl group in the four position did not show improved binding.

Conclusions

Inhibition of uPA by uncharged inhibitor 8b represents a proof of concept that uPA inhibitors without a charged arginino mimetic group can be developed. Inhibitor 8b, which exhibits a dissociation constant in the low nanomolar range comparable to those of known arginino mimetic inhibitors, represents a lead compound for future development of uncharged inhibitors of uPA. The present study did not address the issue of specificity. Many previous studies of uPA inhibitors with arginino mimetic groups attached to various scaffold have resulted in the development of selective inhibitors of uPA. This information should be useful for developing selective uncharged inhibitors of uPA.

Experimental

Modeling

The x-ray crystal structure of human urokinase (pdb code 1EJN) was obtained from the protein data bank. All compounds shown in Table 1 were docked to the enzyme using Autodock 3.0^{21,22} on a cluster of Silicon Graphics workstations consisting of Octanes and O2s. The compounds were prepared using Sybyl 7.0 (Tripos Inc., St. Louis, Mo.). The molecules were drawn in, assigned partial charges using the included Gasteiger-Huckel method and energy minimized using the BFGS method. Minimizations were run for 10,000 iterations and the rotatable bonds defined before docking. The protein was prepared before docking in Sybyl by removing non-native substrates and water molecules. Polar hydrogens and Kollman Uni charges were added to the protein as well. The molecules were docked in an area defined around the active site serine 195 by a cube of 60×60×60 Å using a Lamarckian genetic algorithm.

Chemical Synthesis

Reagent quality solvents were used without purification. Benzoyl chloride was distilled before use. Melting points were determined on a Thomas Hoover capillary melting point apparatus and are uncorrected. NMR spectra were recorded on a Bruker AC250 NMR spectrometer in CDCl₃ unless noted. Chemical shifts are reported in ppm (6) relative to CHCI₃ at 7.24 ppm for ¹H NMR and 77.0 for ¹³C NMR. Chemical shifts are reported in ppm (δ) relative to TMS. Proton NMR peaks are reported as follows: chemical shift, multiplicity (s=singlet, d=doublet, t=triplet, q=quartet, m=multiplet, dd=doublet of doublets and br broad), integration, and coupling constants (J in Hz). High resolution mass spectra were performed at the UNM Mass Spectrometry Facility, University of New Mexico, Albuquerque N.M. Analytical data was obtained from Galbraith laboratories, Knoxville Tenn. 5-Nitrohomophthalic acid was prepared by the method of Ungnade, Nightingale, and French¹⁹. Compounds 3a-3e, 4a-4e, 5c-5e, 6a, 6b, 10a, 10b and 11 were prepared by the method of Kerrigan²³ et al as described for the preparation of compounds 3e, 4e, 5e, 6a, 10a, and 11. Compounds 8a and 8b were prepared according to the method of Kerrigan²³ et al as described for the preparation of 8a. Compounds 7a, 7b, 9a, 9b were prepared according to the method of Kam²⁴ et al as described for 7b and 9b.

2-[2-(2-Bromoethoxy)-2-oxoethyl]-5-nitrobenzoic acid (3a) (66% yield) Tan crystals: mp 113-115° C. [lit.²⁵ 90° C.]; ¹H NMR: δ 3.50 (t, 2H, J=5.96 Hz) 4.21 (s, 2H) 4.43 (t, 2H, J=6.06 Hz) 7.51 (d, 1H, J=8.34 Hz) 8.39 (dd, 1H, J=2.59 Hz, 8.39 Hz) 8.98 (d, J=2.39 Hz); ¹³C NMR: δ 28.40, 40.13, 64.17, 126.24, 126.45, 130.97, 133.32, 142.59, 146.84, 167.46, 169.63.

2-12-(3-Bromopropoxy)-2-oxoethyl]-5-nitrobenzoic acid (3b) (51% yield) White crystals: mp 122-123° C.; ¹H NMR: δ 2.19 (m, 2H) 3.44 (t, 2H, J=6.56 Hz) 4.18 (s, 2H) 4.28 (t, 2H, J=5.96 Hz) 7.51 (d, 1H, J=8.34 Hz) 8.39 (dd, 1H, J=2.39 Hz, 8.35 Hz) 8.98 (d, 1H, J=2.18 Hz) 9.78 (br s, 1H); ¹³C NMR: δ 29.18, 31.58, 40.51, 63.12, 126.84, 127.52, 129.77, 143.37, 147.47, 169.92, 170.05.

2-[2-(2-Bromoethoxy)-2-oxoethyl]benzoic acid (3c) (50% yield) Tan crystals: mp 82-83° C.; ¹H NMR: δ 3.50 (t, 2H, J=6.25 Hz) 4.07 (s, 2H) 4.40 (t, 2H, J=6.25 Hz) 7.27 (d, 1H, J=7.75 Hz) 7.45 (t, 1H, J=7.55 Hz) 7.54 (td, 1H, J=1.39 Hz, 7.55 Hz) 8.14 (d, 1H, J=7.74 Hz); ¹³C NMR: δ 28.50, 40.68, 64.08, 127.65, 128.32, 131.95, 132.43, 133.35, 136.40, 170.86, 172.43.

2-[2-(3-Bromopropoxy)-2-oxoethyl]benzoic acid (3d) (70% yield) White crystals: mp 79-80° C.; ¹H NMR: δ 2.16 (m, 2H, J=6.31 Hz) 3.42 (t, 2H, J=6.55 Hz) 4.05 (s, 2H) 4.24 (t, 2H, J=5.96 Hz) 7.27 (d, 1H, J=7.55 Hz) 7.40 (t, 1H, J=7.65 Hz) 7.54 (td, 1

H, J=1.2, 7.35 Hz) 8.14 (dd, 1H, J=0.99, 7.74 Hz); ¹³C NMR: δ 29.50, 31.80, 40.80, 62.52, 127.60, 128.35, 131.91, 132.41, 133.35, 136.86, 171.17, 172.47.

2-12-(4-Bromobutoxy)-2-oxoethyl]benzoic acid (3e) A solution of 4-bromo-1-butanol (2e, 6.0 mL, 41.6 mmol), homophthalic acid (1c, 2.5 g, 13.8 mmol), and five drops of concentrated sulfuric acid was refluxed in benzene (50 mL) for four hours. The solution was cooled and washed with water (2×25 mL), brine (1×25 mL), and dried over magnesium sulfate. Filtration and evaporation of the solvent gave a dark oil that was triturated with hexane to afford a crude solid. Recrystallization from hexane/ethyl acetate gave 0.91 g (40%) of compound 3e as white crystals: mp 84-86° C.; ¹H NMR: δ 1.79 (m, 2H) 1.87 (m, 2H) 3.38 (t, 2H, J=6.45 Hz) 4.04 (s, 2H) 4.13 (t, 2H, J=6.15 Hz) 7.27 (d, 1H, J=7.55 Hz) 7.39 (t, 2H, J=7.65 Hz) 7.54 (td, 1H, J=1.4, 7.55 Hz) 8.13 (dd, 1H, J=1.19, 7.74 Hz); ¹³C NMR: δ 27.33, 29.32, 33.19, 63.85, 127.56, 128.41, 131.88, 132.42, 133.33, 136.77, 171.32, 172.58. Exact mass calcd for C₂₁H₁₈O₅: 329.9658, observed (M+H) 330.9734.

3-(2-Bromoethoxy)-4-chloro-7-nitroisocoumarin (4a) (43% yield) Yellow crystals: mp 126-128° C. [lit.²⁵ 120° C.]; ¹H NMR: δ 3.67 (t, 2H, J=6.16 Hz) 4.74 (t, 2H, J=6.06 Hz) 7.86 (d, 1H, J=8.94 Hz) 8.53 (dd, 1H, J=2.39 Hz, 8.94 Hz) 9.03 (d, 1H, J=2.28 Hz); ¹³C NMR: δ 27.72, 69.63, 90.86, 117.17, 123.81, 126.32, 129.82, 142.71, 145.47, 154.77, 157.06.

3-(3-Bromopropoxy)-4-chloro-7-nitroisocoumarin (4b) (76% yield) Pale yellow crystals: mp 131-134° C.; ¹H NMR: δ 2.37 (m, 2H) 3.59 (t, 2H, J=6.26 Hz) 4.61 (t, 2H, J=5.86 Hz) 7.81 (d, 1H, J=8.94 Hz) 8.50 (dd, 1H, J=2.09 Hz, 8.84 Hz) 8.99 (d, 1H, J=1.79 Hz); ¹³C NMR: δ 28.49, 31.92, 68.56, 90.46, 116.93, 123.52, 126.22, 129.68, 142.77, 145.77, 155.34, 157.18.

3-(2-Bromoethoxy)-4-chloroisocoumarin (4c) (30% yield) Yellow solid: mp 81-82° C.; ¹H NMR: δ 3.65 (t, 2H, J=6.35 Hz) 4.64 (t, 2H, J=6.35 Hz) 7.41 (t, 1H, J=7.15 Hz) 7.74 (m, 2H) 8.20 (d, 1H, J=7.75 Hz); ¹³C NMR: δ 28.07, 69.37, 92.09, 117.53, 122.47, 126.55, 130.06, 135.62, 137.35, 152.08, 159.01.

3-(3-Bromopropoxy)-4-chloroisocoumarin (4d) (53% yield) Yellow crystals: mp 95-97° C.; ¹H NMR: δ 2.33 (m, 2H) 3.60 (t, 2H, J=6.35 Hz) 4.51 (t, 2H, J=5.76 Hz) 7.40 (t, 1H, J=6.75 Hz) 7.72 (m, 2H) 8.19 (d, 1H, J=7.94 Hz); ¹³C NMR: δ 28.88, 32.26, 68.28, 91.91, 117.58, 122.39, 126.43, 130.12, 135.62, 137.57, 152.74, 159.33.

3-(4-Bromobutoxy)4-chloroisocoumarin (4e) A solution of 3e (0.75 g, 2.3 mmol) and phosphorus pentachloride (1.23 g, 5.9 mmol) was refluxed in benzene (50 mL) for fourteen hours. The orange solution was cooled, washed with water (2×25 mL), saturated sodium bicarbonate (2×15 mL), brine (1×25 mL), and dried over magnesium sulfate. Filtration and evaporation of the solvent gave a yellow oil. Trituration with hexane gave 0.55 g (70%) of compound 4e as white crystals: mp 75-77° C.; ¹H NMR: δ 1.98 (m, 2H) 2.06 (m, 2H) 3.48 (t, 2H, J=6.25 Hz) 4.40 (t, 2H, J=5.96 Hz) 7.38 (td, 1H, J=1.59 Hz, 7.50 Hz) 7.70 (m, 2H) 8.17 (d, 1H, J=7.55 Hz); ¹³C NMR: δ 27.33, 29.32, 33.12, 40.80, 63.81, 127.55, 128.41. 131.86, 132.38, 133.30, 136.76, 171.26, 172.29.

4-Chloro-3-(2-isothioureidoethoxy)isocoumarin hydrobromide (5c) (64% yield) Yellow solid: mp 168-170° C. [lit.²⁴ 167-169° C.]; ¹H NMR: (DMSO-d₆) δ 3.65 (t, 2H, J=5.66 Hz) 4.58 (t, 2H, J=5.67 Hz) 7.53 (t, 1H, J=7.65 Hz) 7.69 (d, 1H, J=8.14 Hz) 7.92 (t, 1H, J=7.05 Hz) 8.13 (d, 1H, J=7.75 Hz) 9.15 (br s, 4H); ¹³C NMR: δ 29.73, 68.11, 90.48, 117.18, 121.72, 126.70, 129.48, 135.99, 136.56, 152.18, 158.35, 169.11.

4-Chloro-3-(3-isothioureidopropoxy)isocoumarin hydrobromide (5d) (40% yield) Yellow solid: mp 159-163° C. [lit.²⁴ 165-167° C.]; ¹H NMR: (DMSO-d₆) 2.21 (m, 2H) 3.40 (t, 2H, J=7.18 Hz) 4.55 (t, 2H, J=6.11 Hz) 7.62 (td, 1H, J=0.95, 7.60 Hz) 7.79 (d, 1H, J=7.70 Hz) 8.02 (td, 1H, J=1.23, 7.70 Hz) 8.23 (dd, 1H, J=1.25, 7.50 Hz) 10.09 (br s, 4H); ¹³C NMR: δ 26.66, 28.53, 68.68, 90.43, 117.13, 121.66, 126.59, 129.47, 135.96, 136.66, 152.63, 158.53, 169.36.

4-Chloro-3-(4-isothioureidobutoxy)isocoumarin hydrobromide (5e) A solution of 4e (0.25 g, 0.75 mmol) and thiourea (0.075 g, 0.98 mmol) in dry tetrahydrofuran (25 mL) was refluxed for forty-eight hours. The resulting pale yellow solid was filtered and washed with hot tetrahydrofuran (3×10 mL) to give 0.2 g (65%) of compound 5h as a pale yellow solid: mp 159-161° C.; ¹H NMR: (DMSO-d₆) δ 1.82 (br s, 4H) 3.24 (t, 2H, J=6.45 Hz) 4.39 (t, 2H, J=5.75 Hz) 7.50 (t, 1H, J=7.45 Hz) 7.65 (d, 1H, 7.45 Hz) 7.89 (t, 1H, J=7.25 Hz) 8.09 (d, 1H, J=7.75 Hz) 9.07 (br s, 4H); ¹³C NMR: δ 24.92, 27.35, 29.59, 69.96, 90.20, 116.95, 121.56, 126.48, 129.45, 135.94, 136.73, 152.80, 158.59, 169.56.

7-Amino-3-(2-bromoethoxy)4-chloroisocoumarin (6a) Compound 4a (2.2 g, 6.3 mmol) was reduced on a Parr apparatus with hydrogen over 10% palladium on charcoal (50 mg) in ethanol (25 mL) until the reaction stopped absorbing hydrogen. The solution was filtered through celite and the filtrate was evaporated. The resulting crude solid was chromatographed (dichloromethane) to give 1.55 g (78%) of compound 6a as yellow crystals: mp 134-136° C., [lit.²⁶ 134-137° C.]; ¹H NMR: δ 3.63 (t, 2H, J=6.46 Hz) 3.95 (br s, 2H) 4.56 (t, 2H, J=6.36 Hz) 7.10 (dd, 1H, J=2.58 Hz, 8.54 Hz) 7.43 (d, 1H, J=2.58 Hz) 7.54 (d, 1H, J=8.74 Hz); ¹³C NMR: δ 28.18, 69.87, 93.59, 113.09, 119.21, 123.54, 124.04, 128.24, 145.63, 149.90, 159.47.

7-Amino-3-(3-bromopropoxy)-4-chloroisocoumarin (6b) (75% yield) Yellow crystals: mp 106-107° C. [lit.^{24,26} 98-100° C.]; ¹H NMR (DMSO-d₆) δ 2.29 (m, 2H) 3.60 (t, 2H, J=6.36 Hz) 4.42 (t, 2H, J=5.76 Hz) 7.09 (dd, 1H, J=2.09 Hz, 8.44 Hz) 7.42 (d, 1H, J=1.99 Hz), 7.51 (d, 1H, J=8.54 Hz); ¹³C NMR: δ 29.11, 32.36, 68.71, 93.35, 113.11, 119.17, 123.58, 123.91, 128.42, 145.56, 150.49, 159.74.

7-Amino-3-(2-isothioureidoethoxy)isocoumarin hydrobromide (7a) (40% yield) Pale yellow solid: mp d 150° C.; ¹H NMR: (DMSO-d₆) δ 3.59 (br s, 2H) 4.47 (br s, 2H) 5.81 (br s, 2H) 7.21 (d, 1H, J=8.94 Hz) 7.26 (s, 1H) 7.44 (br d, 1H) 9.11 (br s, 4H); ¹³C NMR: δ29.73, 68.11, 90.48, 117.18, 121.72, 126.70, 129.48, 135.99, 136.56, 152.18, 158.35, 169.11.

7-Amino-3-(3-isothioureidopropoxy)isocoumarin hydrobromide (7b) A solution of 6b (0.25 g, 0.75 mmol), thiourea (0.071 g, 0.94 mmol) and tetrahydrofuran (25 mL) was refluxed for forty-eight hours to give a yellow precipitate. The precipitate was filtered and washed with hot tetrahydrofuran (3×25 mL), and recrystallized from methanol/ether to give 0.06 g (20%) of compound 7b as a pale yellow solid: mp 173° C.; [lit.²⁴ 160-162° C.]; ¹H NMR: (DMSO-d₆) δ 2.07 (br s, 2H) 3.30 (br s, 2H) 4.32 (br s, 2H) 7.16 (d, 1H, J=7.94 Hz) 7.26 (s, 1H) 7.41 (d, 1H, J=7.95 Hz) 9.05 (br s, 4H); ¹³C NMR: δ 26.73, 28.35, 69.26, 92.87, 110.88, 118.81, 122.84, 123.11, 124.66, 148.23, 149.41, 159.06, 169.37.

7-Benzamido-3-(2-bromoethoxy)-4-chloroisocoumarin (8a) To a solution of 6a (0.75 g, 2.4 mmol) in dry tetrahydrofuran (20 mL) was added benzoyl chloride (0.35 mL, 2.8 mmol) and triethylamine (0.33 mL, 2.3 mmol). The solution was stirred at room temperature for fourteen hours after which time the triethylamine hydrochloride was filtered off and washed with hot tetrahydrofuran (2×10 mL). The filtrate was evaporated to give a pale yellow solid that was recrystallized from tetrahydrofuran/hexane to afford 0.60 g (75%) of compound 8a as a pale yellow solid: mp 214-216° C.; ¹H NMR: (DMSO-d₆) δ 3.83 (t, 2H, J=5.46 Hz) 4.65 (t, 2H, J=5.46 Hz) 7.56 (m, 3H) 7.71 (d, 1H, J=8.93 Hz) 7.99 (d, 2H, J=8.15 ) 8.29 (dd, 1H, J=2.39 Hz, 8.74 Hz) 8.68 (d, 1H, J=2.18 Hz) 10.63 (s, 1H); ¹³C NMR: δ 30.49, 69.98, 91.08, 117.64, 119.23, 122.38, 127.52, 127.86, 128.20, 131.62, 131.90, 134.12, 137.90, 151.40, 158.35, 165.43. Exact mass calcd for C₁₈H₃BrClNO₄: 420.9716, observed (M+H) 421.9788.

7-Benzamido-3-(3-bromopropoxy)-4-chloroisocoumarin (8b) (82% yield) Pale yellow solid: mp 193-194° C.; ¹H NMR: (DMSO-d₆) δ 2.28 (m, 2H) 3.66 (t, 2H, J=6.56 Hz) 4.44 (t, 2H, J=5.96 Hz) 7.55 (m, 3H) 7.68 (d, 1H, J=8.74 Hz) 7.98 (d, 6.56 Hz) 8.25 (dd, 1H, J=1.99 Hz, 8.74 Hz) 8.66 (d, 1H, J=1.98 Hz) 10.62 (s, 1H); 13C NMR: δ 30.74, 32.09, 69.13, 91.36, 118.01, 119.74, 122.80, 127.88, 128.43, 128.67, 133.10, 132.46, 134.47, 138.15, 152.20, 158.95, 165.96. Exact mass calcd for C₁₉H₁₅BrClNO₄: 434.9873, observed (M+H) 435.9959.

7-Benzamido-4-chloro-3-(2-isothioureidoethoxy)isocoumarin hydrobromide (9a) A solution of 8a (0.3 g, 0.71 mmol) and thiourea (0.06 g, 0.78 mmol) in dry tetrahydrofuran (25 mL) was refluxed for twelve hours. The resulting pale yellow solids were filtered and washed with hot tetrahydrofuran (3×10 mL) to give 0.06 g (17%) of compound 9a as a pale yellow solid: mp 173-175° C. Evaporation of the filtrate afforded 8a (0.2 g). Yield based on recovered starting material is 51%; ¹H NMR: (DMSO-d₆) δ 3.68 (br s, 2H) 4.60 (br s, 2H) 7.59 (m, 3H) 7.74 (d, 1H, J=8.54 Hz) 8.03 (d, 2H, J=6.75 Hz) 8.32 (d, 1H, J=8.14 Hz) 8.73 (s, 1H) 9.18 (br s, 4H) 10.69 (s, 1H); ¹³C NMR: δ 29.85, 68.32, 91.00, 117.72, 119.47, 122.53, 127.60, 128.22, 128.33, 131.79, 132.06, 134.12, 137.96, 151.44, 158.43, 165.63, 169.18.

7-Benzamido-4-chloro-3-(3-isothioureidopropoxy)isocoumarin hydrobromide (9b) (25% yield) Pale yellow solid: mp 203-204° C.; ¹H NMR: (DMSO-d₆) δ 2.12 (m, 2H) 3.31 (br s, 2H) 4.44 (t, 2H, J=5.66 Hz) 7.56 (m, 3H) 7.71 (d, 1H, J=8.74 Hz) 8.00 (d, 2H, J=6.95 Hz) 8.28 (d, 1H, J=8.54 Hz) 8.70 (s, 1H) 9.07 (br s, 4H) 10.65 (s, 1H); ¹³C NMR: δ 27.21, 28.79, 69.37, 91.38, 118.13, 119.85, 122.92, 128.04, 128.60, 128.77, 132.21, 132.57, 134.61, 138.33, 152.36, 159.05, 166.02, 169.83.

3-(2-Bromoethoxy)-7-nitroisocoumarin (10a) A solution of 3a (1.5 g, 4.5 mmol) and trifluoroacetic anhydride (0.64 mL, 5.0 mmol) in dichloromethane (50 mL) was stirred at room temperature for sixteen hours. The solution was evaporated, washed with water (1×25 mL), saturated sodium bicarbonate solution (1×25 mL), dried over magnesium sulfate, and evaporated to afford 1.23 g (87%) of a crude yellow solid. Recrystallization from isopropanol gave 0.66 g (47%) of compound 10a as yellow crystals: mp 95-97° C.; ¹H NMR: δ 3.65 (t, 2H, J=5.96 Hz) 4.54 (t, 2H, J=5.86 Hz) 5.77 (s, 1H) 7.42 (d, 1H, J=8.74 Hz) 8.38 (d, 1H, J=8.54 Hz) 8.96 (s, 1H); ¹³C NMR: δ 27.51, 68.87, 81.12, 117.12, 125.70, 126.17, 129.28, 144.97,145.11, 158.81, 160.02.

3-(3-Bromopropoxy)-7-nitroisocoumarin (10b) (55% yield) Light tan solid: mp 112-113° C.; ¹H NMR: δ 2.37 (m, 2H) 3.59 (t, 2H, J=6.26 Hz) 4.38 (t, 2H, J=5.86 Hz) 5.72 (s, 1H) 7.43 (d, 1H, J=8.74 Hz) 8.41 (dd, 1H, J=2.48 Hz, 8.84 Hz) 9.02 (d, 1H, J=2.19 Hz); ¹³C NMR: δ 28.80, 31.46, 67.29, 80.02, 117.02, 125.55, 126.07, 129.15, 144.79, 145.21, 158.94, 160.89.

3-(3-Bromopropoxy)-4-trifluoroacetylisocoumarin (10c) A solution of 3d (0.60 g, 2.0 mmol) and trifluoroacetic anhydride (0.38 mL, 2.7 mmol) in dichloromethane (25 mL) was stirred at room temperature for fourteen hours. The solution was evaporated and the oil was chromatographed (chloroform) to afford 0.45 g (59%) of compound 10c as white crystals: mp 1 6-117° C.; ¹H NMR: δ 2.38 (m, 2H) 3.54 (t, 2H, J=6.26 Hz) 4.70 (t, 2H, J=5.96 Hz) 7.42 (m, 1H) 7.74 (m, 1H) 8.10 (d, 1H, J=8.34 Hz) 8.22 (d, 1H, J=7.95 Hz); ¹³C NMR: δ 28.24, 31.37, 68.94, 90.90, 115.81, 116.01, 123.40, 126.75, 130.26, 135.87, 136.27, 157.73, 162.08, 179.97. Anal. Calcd. for C₁₄H₁₀BrF₃O₄: C, 44.35; H, 2.66. Found: C, 44.25; H, 2.99.

7-Amino-3-(2-bromoethoxy)isocoumarin (11) A solution of 10a (1.5 g, 4.7 mmol) in methanol/ethyl acetate (1:1, 25 mL) was reduced on a Parr apparatus with hydrogen and 10% palladium on charcoal. After the reaction stopped absorbing hydrogen it was filtered through celite and the celite was washed with methylene chloride (3×50 mL). The filtrate was evaporated to near dryness keeping the temperature below 40° C. The semisolid was recrystallized from methylene chloride/methanol to afford 1.10 g (81%) of compound 11 as yellow crystals: mp>280° C.; ¹H NMR: (DMSO-d₆) 3.79 (t, 2H, J=5.17 Hz) 4.36 (t, 2H, J=4.97 Hz) 5.50 (s, 2H) 5.82 (s, 1H) 7.03 (d, 1H, J=8.54 Hz) 7.19 (m, 2H); ¹³C NMR: δ30.30, 68.71, 80.35, 110.20, 117.91, 123.17, 125.84, 128.03, 147.16, 154.71, 160.45.

Kinetics

Human urokinase (Sigma/Aldrich, St. Louis, Mo.) and Spectrozyme UK (American Diagnostica, Stamford, Conn.) were used for the kinetic studies. Enzyme activity was routinely measured in 1 ml volumes of 0.1M Tris, pH 8.8, Spectrozyme UK (10 μM to 150 μM) and 0.64 μg (3,770 units/mg protein) human urokinase. Reactions were monitored at 405 nm, 25° C., with a Perkin/Elmer Lambda S2 UV/vis spectrophotometer. Michaelis constants and Kᵢ values were determined from initial rate data, measured at 8 to 10 substrate concentrations, by non-linear regression analysis with SigmaPlot's Enzyme Kinetics Module™ (Chicago, Ill., USA).

### REFERENCES

1. Kontogiorgis, C. A.; Papaioannou, P.; Hadjipavlou-Litina, D. J. Curr. Med. Chem. 2005, 12, 339.
2. Lee, M.; Fridman, R.; Mobashery, S. Chem. Soc. Rev. 2004, 33, 401.
3. McIntyre, J. O.; Matrisian, L. M. J. Cell Biochem. 2003, 90, 1087.
4. Duffy, M. J. Curr. Pharm. Des. 2004, 10, 39.
5. Podgorski, I.; Sloane, B. F. Biochem. Soc. Symp. 2003, 70, 263.
6. Vihinen, P.; Kahari, V. M. Int. J. Cancer 2002, 99, 157.
7. Wang, Y. Med. Res. Rev. 2001, 21, 146.
8. Romer, J.; Nielsen, B. S.; Ploug, M. Curr. Pharm. Des. 2004, 10, 2359.
9. Jo, M.; Thomas, K. S.; Marozkina, N.; Amin, T. J.; Silva, C. M.; Parsons, S. J.; Gonias, S. L. J. Biol. Chem. 2005, in press (available online).
10. Steinmetzer, T. IDrugs 2003, 6, 138.
11. Rockway, T. W.; Giranda, V. L. Curr. Pharm. Des. 2003, 9, 1483.
12. Renatus, M.; Bode, W.; Huber, R.; Sturzebecher, J.; Stubbs, M. T. J. Med. Chem. 1998, 41, 5445.
13. Wendt, M. D.; Rockway, T. W.; Geyer, A.; McClelland, W.; Weitzberg, M.; Zhao, X.; Mantei, R.; Nienaber, V. L.; Stewart, K.; Klinghofer, V.; Giranda, V. L. J. Med. Chem. 2004, 47, 303.
14. Mackman, R. L.; Katz, B. A.; Breitenbucher, J. G.; Hui, H. C.; Verner, E.; Luong, C.; Liu, L.; Sprengeler, P. A. J. Med Chem. 2001, 44, 3856.
15. Sperl, S.; Jacob, U.; Arroyo de Prada, N.; Sturzebecker, J.; Wilhelm, O. G.; Bode, W.; Magdolen, V.; Huber, R.; Moroder, L. Proc. Natl. Acad. Sci. 2000, 97, 5113.
16. Barber, C. G.; Dickinson, R. P.; Fish, P. V. Bioorg Med. Chem. Lett. 2004, 14, 3227.
17. Kam, C-M.; Kerrigan, J. E.; Plaskon, R. R.; Duffy, E. J.; Lollar, P.; Suddath, F. L.; Powers, J. C. J. Med. Chem. 1994, 37, 1298.
18. Bryson, H.; Bunning, R. A. D.; Feltell, R.; Kam, C-M.; Kerrigan, J.; Powers, J. C.; Buttle, D. J. Arch. Biochem. Biophys. 1998, 355, 15.
19. Ungnade, H. E.; Nightingale, D. V.; French, H. E. J. Org. Chem. 1945, 10, 533.
20. Devi A. R.; Rajaram S. Indian J. Chem. 2000, 39B, 294.
21. Morris, G. M.; Goodsell, D. S.; Halliday, R. S.; Huey, R.; Hart, W. E.; Belew, R. K.; Olson, A. J. J. Comp. Chem. 1998, 19, 1639.
22. Morris, G. M.; Goodsell, D. S.; Huey, R.; Olson, A. J. J. Comput-Aided Mol. Des. 1996, 10, 293.
23. Kerrigan, J. E.; Oleksyszyn, J.; Kam, C-M; Selzler, J.; Powers, J. C. J. Med. Chem. 1995, 38, 544.
24. Kam, C-M; Fujikawa, K.; Powers, J. C. Biochemistry 1988, 27, 2547.
25. Bihel, F.; Quelever, G.; Lelouard, H.; Petit, A.; Alves da Costa, C.; Pourquie, O.; Checler, F.; Thellend, A.; Pierre, P.; Kraus, J-L. Bioorg. Med. Chem. 2003, 11, 3141.
26. Powers, J. C.; Oleksyszyn, J.; Narasimhan, S. L.; Kam, C-M Biochemistry 1990, 29, 3108.

1. A compound according to the structure: 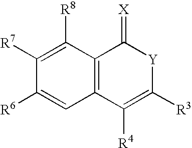
Where X is O or S, preferably O;
Y is O, S, or N, preferably O;
R is an optionally substituted C₁-C₇ alkyl group, an optionally substituted (CH₂)ₙR^{b} group or an OR group;
R^{b} is a guanidino group or a thioguanidino group;
R is an optionally substituted C₁-C₇ alkyl group or an optionally substituted (CH₂)ₙR′ group; n is 0, 1, 2, 3, 4, 5, 6, or 7 (preferably 2, 3 or 4);
R′ is F, Cl, Br or I (preferably Br), NO₂, an R″ group, an OR″ group or an SR″ group, where R″ is an optionally substituted C₁-C₆ alkyl group, a guanidino group or a thioguanidino group;
R⁴ is H, F, Cl, Br, I, NO₂, OH, R¹ or OR¹, where R¹ is an optionally substituted C₁-C₇ alkyl group or an optionally substituted C₂-C₁₁ acyl group;
R⁶ is H, an optionally substituted C₁-C₆ alkyl group, or together with R⁷ forms an optionally substituted 5-7 membered saturated or unsaturated carbocyclic group, an optionally substituted 5-7 membered saturated or unsaturated heterocyclic group, or an optionally substituted aromatic or heteroaromatic group;
R⁷ is H, F, Cl, Br, I, NO₂, NR^{a′}R^{b′} or NHR^{b}, where R^{a′} and R^{b′} are independently H or a C₁-C₃ alkyl group and R^{b} is a C₂-C₁₁ acyl group which is optionally substituted, or together with R⁶ or R⁸ forms an optionally substituted 5-7 membered saturated or unsaturated carbocyclic group, an optionally substituted 5-7 membered saturated or unsaturated heterocyclic group, or an optionally substituted aromatic or heteroaromatic group;
R⁸ is H, an optionally substituted C₁-C₆ alkyl group, or together with R⁷ forms an optionally substituted 5-7 membered saturated or unsaturated carbocyclic group, an optionally substituted 5-7 membered saturated or unsaturated heterocyclic group, or an optionally substituted aromatic or heteroaromatic group; and pharmaceutically acceptable salts, thereof.

2. The compound according to claim 1 which is uncharged.

3. A pharmaceutical composition comprising an effective amount of a compound according to claim 1, optionally in combination with a pharmaceutically acceptable carrier, additive or excipient.

4. A method of treating a tumor, including a cancerous tumor in a patient, comprising administering to said patient in need thereof an effective amount of a compound according to claim 1.

5. A method of treating cancer in a patient comprising administering to said patient an effective amount of a compound according to claim 1.

6. The method according to claim 5 wherein said cancer is stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, melanoma, acute lymphocytic leukemia, acute myelogenous leukemia, Ewing's sarcoma, small cell lung cancer, choriocarcinoma, rhabdomyosarcoma, Wilms' Tumor, neuroblastoma, hairy cell leukemia, mouth/pharynx, oesophagus, larynx, kidney or lymphoma.

7. A method of inhibiting urokinase in a patient comprising exposing said to an effective amount of a compound according to claim 1.

8. A method of treating a disease or condition in a patient which is mediated through an angiogenesis mechanism comprising administering to said patient an effective amount of a compound according to claim 1.

9. A method of treating a disease state or condition selected from the group consisting of
retinopathy, age-related macular degeneration, psoriasis, venous ulcers, acne, rosacea, warts, eczema, hemangiomas and lymphangiogenesis, Sturge-Weber syndrome, neurofibromatosis, tuberous sclerosis, chronic inflammatory disease and arthritis comprising administering to said patient an effective amount of a compound according to claim 1 to said patient.
